(19) United States
(12) Patent Application Publication
(10) Pub. No.: US 2026/0283996 A1
(43) Pub. Date: Sep. 24, 2026

(54) DECANOIC ACID AND ITS COMPOSITIONS FOR USE IN THE PREVENTION AND/OR TREATMENT OF PEROXISOMAL DISORDERS

(71) Applicants: SOCIÉTÉ DES PRODUITS NESTLÉ S.A., Vevey (CH); UCL BUSINESS LTD, London (GB)

(72) Inventors: Simon James Robert HEALES, Maidenhead (GB); Simon James EATON, London (GB); Tomas BALDWIN, Hanwell (GB)

(21) Appl. No.: 19/472,921

(22) PCT Filed: Apr. 12, 2024
(86) PCT No.: PCT/EP2024/059977

§ 371 Date: Oct. 6, 2025

(30) Foreign Application Priority Data
Apr. 14, 2023 (EP) 23167980.4

(51) Int. Cl. A61K 31/23; A61K 9/00; A61K 9/107; A61P 3/00

(52) U.S. Cl. CPC A61K 31/23; A61K 9/0053; A61K 9/107; A61P 3/00

(57) ABSTRACT

The present invention relates to decanoic acid and compositions comprising the same for treating and/or preventing a peroxisomal disorder.

### FIELD OF THE INVENTION

The present invention relates to decanoic acid and compositions comprising the same for treating and/or preventing a peroxisomal disorder.

### BACKGROUND TO THE INVENTION

Peroxisomes are small membrane-enclosed organelles that are indispensable to human health and development. Peroxisomes contain enzymes involved in a variety of metabolic reactions, such as β-oxidation of fatty acids, bio-synthesis of ether phospholipids and metabolism of reactive oxygen species (Islinger, M., et al., 2012. Histochemistry and cell biology, 137 (5), pp. 547-574).

Peroxisomal disorders are a group of medical conditions characterised by dysfunction of peroxisomes. Defects in genes encoding peroxisomal proteins may lead to a variety of diseases that can be grouped into two main classes of peroxisomal disorders based on the cause and consequence of the defects, the single peroxisomal enzyme deficiencies (PEDs) and the peroxisome biogenesis disorders (PBDs), including Zellweger Spectrum Disorders (ZSDs). Taken as a group, the peroxisomal disorders constitute a substantial fraction of the inborn errors of metabolism and have an estimated combined incidence of 1 in 5000 (Waterham, H. R., et al., 2016. BBA-Molecular Cell Research, 1863 (5), pp. 922-933).

For many of the peroxisomal disorders, there is no standard course of treatment, with treatment strategies focusing on symptomatic or supportive therapies. Currently, there is no specific diet that is recommended for PBD-ZSD patients (Braverman, N. E., et al., 2016. Molecular genetics and metabolism, 117 (3), pp. 313-321).

Thus, there is a demand for new strategies for treating and/or preventing peroxisomal disorders.

### SUMMARY OF THE INVENTION

The inventors have surprisingly found that decanoic acid may exert positive effects upon peroxisomal content and function. These positive effects support the application of decanoic acid or compositions comprising the same for treating and/or preventing a peroxisomal disorder.

In one aspect, the present invention provides decanoic acid for use in treating and/or preventing a peroxisomal disorder.

In another aspect, the present invention provides a method of treating and/or preventing a peroxisomal disorder, the method comprising administering a therapeutically effective amount of decanoic acid to a subject in need thereof.

In another aspect, the present invention provides the use of decanoic acid for the manufacture of a medicament for the treatment and/or prevention of a peroxisomal disorder.

In another aspect, the present invention provides the use of decanoic acid as a peroxisome proliferator, optionally in vitro.

The peroxisomal disorder may be any medical condition characterised by dysfunction of peroxisomes. Suitably, the peroxisomal disorder is a peroxisome biogenesis disorder (PBD) or a peroxisomal enzyme deficiency (PED). Suitably, the peroxisomal disorder is a Zellweger spectrum disorder (ZSD); rhizomelic chondriodysplasia punctata (RCDP) type 1; RCDP type 5; x-linked adrenoleukodystrophy (X-ALD); acyl-CoA oxidase deficiency; D-bifunctional protein deficiency; SCPx deficiency; 2-methylacyl-CoA racemace (AMACR) deficiency; Refsum disease; primary hyperoxaluria type 1; RCDP type 2; RCDP type 3; RCDP type 4; ABCD3 deficiency; BAAT deficiency; or Acatalasemia. In some embodiments, the peroxisomal disorder is a peroxisome biogenesis disorder (PBD). In some embodiments, the peroxisomal disorder is a Zellweger spectrum disorder (ZSD), rhizomelic chondriodysplasia punctata (RCDP) type 1, or RCDP type 5. In some embodiments, the peroxisomal disorder is a ZSD. In some embodiments, the peroxisomal disorder is Heimler syndrome (HS), infantile Refsum disease (IRD), neonatal adrenoleukodystrophy (NALD), or Zellweger syndrome (ZS). In some embodiments, the peroxisomal disorder is HS, IRD or NALD.

The decanoic acid may be used to treat and/or prevent a peroxisomal disorder in any subject in need thereof. Suitably, the subject is a mammal. Suitably, the subject is a human. Suitably, the subject is a child or an adult. Suitably, the subject has a defect in a PEX gene or a gene encoding a peroxisomal protein. Suitably, the subject has a defect in one or more gene selected from: PEX1, PEX2, PEX3, PEX5, PEX6, PEX7, PEX10, PEX11B, PEX12, PEX13, PEX14, PEX16, PEX19, PEX26, ABCD1, ACOX1, HSD17B4, SCP2, AMACR, PHYH, AGXT, GNPAT, AGPS, FAR1, ABCD3, BAAT, and CAT. In some embodiments, the subject has a defect in a PEX gene. In some embodiments, the subject has a defect in one or more gene selected from: PEX1, PEX2, PEX3, PEX5, PEX6, PEX7, PEX10, PEX11B, PEX12, PEX13, PEX14, PEX16, PEX19, PEX26.

The decanoic acid may be provided in any suitable form. Suitably, the decanoic acid is in the form of triglycerides, diacyl-glycerides, monoacyl-glycerides, a pharmaceutically acceptable salt, and/or an ester. In some embodiments, the decanoic acid is in the form of triglycerides, diacyl-glycerides, and/or monoacyl-glycerides. In some embodiments, the decanoic acid is in the form of triglycerides. In some embodiments, the decanoic acid is in the form of medium-chain triglycerides. In some embodiments, the decanoic acid is in the form of a C10 homotriglyceride.

The decanoic acid may be comprised in a composition. The composition may be any suitable composition. Suitably, the composition is a pharmaceutical composition, a nutritional composition, or a nutritional supplement. Suitably, the composition is in the form of an oil-in-water emulsion, a powder, or a foodstuff. In some embodiments, the composition is in the form of an oil-in-water emulsion. In some embodiments, the composition is in a ready-to-use form.

The decanoic acid may be present in the composition in any suitable amount and in combination with other suitable components. Suitably, at least about 50 wt % of the fat is decanoic acid. In some embodiments, at least about 55 wt %, at least about 60 wt %, at least about 65 wt %, or at least about 70 wt % of the fat is decanoic acid. Suitably, the decanoic acid is present in the composition in an amount of at least about 100 g/L. In some embodiments, the decanoic acid is present in the composition in an amount of at least about 110 g/L, at least about 120 g/L, at least about 130 g/L, at least about 140 g/L, at least about 150 g/L, at least about 160 g/L, at least about 180 g/L, at least about 200 g/L, at least about 220 g/L, or at least about 240 g/L. In some embodiments, the composition is free from or substantially free from fatty acid moieties that are not decanoic acid or octanoic acid. In some embodiments, the composition is free from or substantially free from fatty acid moieties that are not decanoic acid. In some embodiments, the composition is free from MCTs comprising fatty acid moieties that are not decanoic acid or octanoic acid. In some embodiments, the composition is free from or substantially free from MCTs comprising fatty acid moieties that are not decanoic acid. Suitably, the composition comprises proteins, fat and carbohydrates.

The decanoic acid may be administered by any suitable administration route and in any suitable administration regimen. Suitably, the decanoic acid is administered enterally or parenterally. In some embodiments, the decanoic acid is administered enterally. In some embodiments, the decanoic acid is administered orally or via feeding tube. Suitably, the decanoic acid is administered once a day, twice a day, three times a day, or four times a day. In some embodiments, the decanoic acid is administered as part of a meal, snack or enteral feed. Suitably, the decanoic acid is administered in an amount of about 2 g to about 200 g per day, about 5 g to about 150 g per day, or about 10 g to about 100 g per day.

The decanoic acid may improve peroxisomal function and/or peroxisomal number. The decanoic acid may improve peroxisomal function. For example, the decanoic acid may increase acyl CoA oxidase activity, increase Pex11a expression, and/or increase peroxisomal beta oxidation capacity.

### DESCRIPTION OF DRAWINGS

FIG. 1—Effect of decanoic acid on peroxisomal content and function

SH-SY5Y cells were exposed to 250 μM C10 or C8 for 6 days. The following parameters were studied: (A) activity of acyl CoA oxidase; (B) expression of PEX11a; and (C) evaluation of peroxisomal fatty acid beta oxidation.

### DETAILED DESCRIPTION

Various preferred features and embodiments of the present invention will now be described by way of non-limiting examples. This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The publications discussed herein are provided solely for their disclosure prior to the filing date of the present application. Nothing herein is to be construed as an admission that such publications constitute prior art to the claims appended hereto.

The practice of the present invention will employ, unless otherwise indicated, conventional techniques which are within the capabilities of a person of ordinary skill in the art. All publications mentioned in the specification are herein incorporated by reference.

### Decanoic Acid and Compositions Comprising the Same

The present invention relates to decanoic acid and compositions comprising the same for treating and/or preventing a peroxisomal disorder.

### Decanoic Acid

Decanoic acid (also known as capric acid) is a saturated fatty acid of the formula CH₃(CH₂)₈COOH. Decanoic acid may be in free form (or a salt or ester thereof) or in the form of, for example, triglycerides, diacyl-glycerides, or monoacyl-glycerides.

In some embodiments, the decanoic acid is in the form of triglycerides, diacyl-glycerides, and/or monoacyl-glycerides. In preferred embodiments, the decanoic acid is in the form of triglycerides.

In some embodiments, the decanoic acid is in the form of medium-chain triglycerides. A “medium-chain triglyceride” (MCT) may refer to a triglyceride in which all three fatty acid moieties are medium-chain fatty acid moieties. The term “fatty acid moiety” may refer to the part of the MCT that originates from a fatty acid in an esterification reaction with glycerol. For example, an esterification reaction between glycerol and only decanoic acid would result in a MCT with decanoic acid moieties. Suitably, medium-chain fatty acids (MCFA) are fatty acids that have 6 to 12 carbon atoms, although fatty acids with 8 and 10 carbon atoms (i.e. octanoic acid and decanoic acid) are preferred and may be referred to herein as C8 fatty acids or C8, and C10 fatty acids or C10.

Homotriglycerides (i.e. all of the fatty acid moieties of the triglyceride are of the same identity, for example a C10 homotriglyceride may comprise 3 decanoic acid moieties) and/or heterotriglycerides (i.e. the fatty acid moieties of the MCT are not all the same identity) may be used in the present invention. Preferred heterotriglycerides are heterotriglycerides made up of octanoic acid and decanoic acid moieties. In some embodiments, the decanoic acid is in the form of a C10 homotriglyceride. In some embodiments, the decanoic acid is in the form of a C8/C10 heterotriglyceride.

The decanoic acid may be obtained by any suitable method known in the art. Examples of natural sources of MCT include plant sources such as coconuts, coconut oil, palm kernels, palm kernel oils, and animal sources such as milk. Decanoic acid forms about 5-8 wt % of the fatty acid composition of coconut oil. MCTs may also be synthesised by esterification of glycerol with one or more medium-chain fatty acids (MCFA). For example, MCT-C10 can be synthesised by esterification of glycerol with decanoic acid.

Alternatively the decanoic acid referred to herein may be in the form of a pharmaceutically acceptable salt or an ester. Salts and esters of decanoic acid are also known in the art as decanoates or caprates.

The decanoic acid or a composition comprising the same may be provided in any suitable form, such as in the form of an oil-in-water emulsion, a powder, or a foodstuff.

In one embodiment, the decanoic acid or a composition comprising the same is in the form of an oil-in-water emulsion. The emulsion may comprise substantially no protein and/or carbohydrate. Suitably, the total fat content of the oil-in-water emulsion is about 5 to about 40 g/100 ml. In one embodiment, the total fat content of the oil-in-water emulsion is about 10 to about 40 g/100 ml, about 15 to about 40 g/100 ml, about 20 to about 40 g/100 ml, about 25 to about 40 g/100 ml, about 30 to about 40 g/100 ml, or about 34 g/100 ml. In other embodiments, the total fat content of the oil-in-water emulsion is about 5 to about 30 g/100 ml, about 5 to about 25 g/100 ml, about 10 to about 25 g/100 ml, about 10 to about 20 g/100 ml or about 15 to about 25 g/100 ml. Suitably, the energy value of the emulsion is about 50 to about 500 kcal per 100 ml. In one embodiment, the energy value of the emulsion is about 100 to about 500 kcal per 100 ml, about 200 to about 500 kcal per 100 ml, about 200 to about 400 kcal per 100 ml, about 250 to about 350 kcal per 100 ml, or about 308 kcal per 100 ml. In other embodiments, the energy value of the emulsion is about 50 to about 300 kcal per 100 ml, for example, about 100 to about 300 kcal per 100 ml, about 50 to about 200 kcal per 100 ml, about 150 to about 250 kcal per 100 ml or about 160 to about 200 kcal per 100 ml.

In one embodiment, the decanoic acid or a composition comprising the same is in the form of a tablet, dragee, capsule, gel cap, powder, granule, solution, emulsion, suspension, coated particle, spray-dried particle or pill.

In one embodiment, the decanoic acid or a composition comprising the same may be in the form of a powder. The powder may, for example, be a spray-dried powder or a freeze-dried powder. In one embodiment, the decanoic acid or a composition comprising the same is in a spray dried form.

The decanoic acid or a composition comprising the same may be provided in a ready-to-use form or in a form suitable for reconstitution in water. In preferred embodiments, the decanoic acid or composition comprising the same is in a ready-to-use form. Suitably, the decanoic acid or composition comprising the same is provided in packs of about 50 mL to about 200 mL, about 80 mL to about 150 mL, or about 120 mL in volume. The decanoic acid or composition comprising the same may be inserted or mixed into a food substance. The decanoic acid or composition comprising the same may be in the form of a food stuff or a feed. In one embodiment, the food stuff is a human food stuff. In one embodiment, the decanoic acid is comprised within a fortifying food or drink.

### Compositions

The decanoic acid (e.g. in the form of triglycerides) may be in the form of a composition.

The composition can be any type of composition in which the decanoic acid can be incorporated, such as a composition in the form of a medical food, a tube feed, a nutritional composition, a nutritional supplement, a complete nutritional product, or a pharmaceutical composition.

The decanoic acid may be formulated for human consumption. The decanoic acid or composition comprising the same may be formulated for enteral or parenteral administration. In a preferred embodiment, the composition is formulated for oral administration.

The decanoic acid or composition comprising the same may be in the form of a medical food. The term “medical food” as used herein may refer to a food product specifically formulated for the dietary management of a medical disease or condition; for example, the medical disease or condition may have distinctive nutritional needs that cannot be met by normal diet alone. The medical food may be administered under medical supervision. The medical food may be for oral ingestion or tube feeding.

The decanoic acid or composition comprising the same may be in the form of a tube feed. The term “tube feed” may refer to a product which is intended for introducing nutrients directly into the gastrointestinal tract of a subject by a feeding tube. A tube feed may be administered by, for example, a feeding tube placed through the nose of a subject (such as nasogastric, nasoduodenal, and nasojejunal tubes), or a feeding tube placed directly into the abdomen of a subject (such as gastrostomy, gastrojejunostomy, or jejunostomy feeding tube).

The decanoic acid or composition comprising the same may be in the form of a nutritional composition or a nutritional supplement. The term “nutritional composition” may refer to a composition which nourishes a subject. The term “nutritional supplement” may refer to a product which is intended to supplement the general diet of a subject. The decanoic acid or composition comprising the same may be in the form of a complete nutritional product. The term “complete nutritional product” may refer to a product which is capable of being the sole source of nourishment for the subject.

The decanoic acid or composition comprising the same may be in the form of a beverage, mayonnaise, salad dressing, margarine, low fat spread, dairy product, cheese spread, processed cheese, dairy dessert, flavoured milk, cream, fermented milk product, cheese, butter, condensed milk product, ice cream mix, soya product, pasteurised liquid egg, bakery product, confectionary product, confectionary bar, chocolate bar, high fat bar, liquid emulsion, spray-dried powder, freeze-dried powder, UHT pudding, pasteurised pudding, gel, jelly, yoghurt, or a food with a fat-based or water-containing filling. The decanoic acid or composition comprising the same may be used to coat a food.

The decanoic acid or composition comprising the same may in the form of a pharmaceutical composition. A “pharmaceutical composition” may refer to a composition that comprises or consists of a therapeutically effective amount of a pharmaceutically active agent i.e. the decanoic acid. A pharmaceutical composition preferably includes a pharmaceutically acceptable carrier, diluent or excipient (including combinations thereof).

Acceptable carriers, diluents and excipients for therapeutic use are well known in the pharmaceutical art. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s) or solubilising agent(s). Nutritionally acceptable carriers, diluents and excipients include those suitable for human or animal consumption and that are used as standard in the food industry. Typical nutritionally acceptable carriers, diluents and excipients will be familiar to the skilled person in the art.

Examples of suitable carriers include lactose, starch, glucose, methyl cellulose, magnesium stearate, mannitol, sorbitol and the like. Examples of suitable diluents include ethanol, glycerol and water. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like.

In addition to the decanoic acid, the composition may comprise any further components in any suitable amount. The composition may comprise proteins, fat, and/or carbohydrates.

In one embodiment, at least about 50 wt % of the fat in the composition is decanoic acid. In one embodiment, at least about 51 wt %, at least about 52 wt %, at least about 53 wt %, at least about 54 wt %, at least about 55 wt %, at least about 56 wt %, at least about 57 wt %, at least about 58 wt %, at least about 59 wt %, at least about 60 wt %, at least about 65 wt %, at least about 70 wt %, at least about 75 wt %, at least about 80 wt %, at least about 85 wt %, at least about 90 wt %, at least about 95 wt %, at least about 98 wt %, at least about 99 wt %, or at least about 100 wt % of the fat in the composition is decanoic acid. In one embodiment, at least about 70 wt % of the fat in the composition is decanoic acid.

In one embodiment, the decanoic acid is in the form of medium chain triglycerides wherein said triglycerides make up at least about 50 wt %, at least about 51 wt %, at least about 52 wt %, at least about 53 wt %, at least about 54 wt %, at least about 55 wt %, at least about 56 wt %, at least about 57 wt %, at least about 58 wt %, at least about 59 wt %, at least about 60 wt %, at least about 70 wt %, at least about 80 wt %, at least about 85 wt %, at least about 90 wt %, at least about 95 wt %, at least about 99 wt %, or 100 wt % of the total fat content of the composition. In one embodiment, the decanoic acid is in the form of medium chain triglycerides wherein said triglycerides make up at least about 70 wt % of the total fat content of the composition.

In one embodiment, decanoic acid is present in the composition in an amount of at least about 5 g/L, at least about 10 g/L, at least about 15 g/L, at least about 20 g/L, at least about 30 g/L, at least about 40 g/L, at least about 50 g/L, at least about 60 g/L, at least about 70 g/L, at least about 80 g/L, at least about 90 g/L, at least about 100 g/L, at least about 110 g/L, at least about 120 g/L, at least about 130 g/L, at least about 140 g/L, at least about 150 g/L, at least about 160 g/L, at least about 180 g/L, at least about 200 g/L, at least about 220 g/L, at least about 240 g/L, at least about 260 g/L, at least about 280 g/L, at least about 300 g/L, at least about 400 g/L, or at least about 500 g/L. In one embodiment, decanoic acid is present in the composition in an amount of at least about 240 g/L. In one embodiment, the composition comprises decanoic acid in an amount of about 100 g/L to about 500 g/L, about 150 g/L to about 400 g/L, or about 200 g/L to about 300 g/L. In one embodiment, the composition comprises decanoic acid in an amount of about 245 g/L.

The composition may comprise total saturated fatty acids in an amount of about 23 g to about 50 g, about 25 g to about 45 g, or about 33 to about 44 g per 100 g lipids, on fatty acid basis. The composition may comprise total saturated fatty acids in an amount of about 27 g to about 35 g, about 28 g to about 34 g, about 29 g to about 33 g, or about 30 g to about 32 g per 100 mL. In one embodiment, the composition comprises total saturated fatty acids in an amount of about 31 g per 100 mL. It is preferred that a major part of the saturated fatty acids is decanoic acid (C10:0). In one embodiment, the composition comprises decanoic acid in an amount of about 15 g to about 50 g, about 18 g to about 45 g, or about 23 g to about 44 g per 100 g lipids, or about 30 g to about 37 g per 100 g lipids.

In one embodiment, the composition is free from or substantially free from fatty acid moieties that are not decanoic acid or octanoic acid. In one embodiment, the composition is free from or substantially free from fatty acid moieties that are not decanoic acid. However, there may be traces of such fatty acid moieties in the composition (e.g., less than about 5 wt %, less than about 4 wt %, less than about 3 wt %, less than about 2 wt %, less than about 1 wt %, less than about 0.5 wt % or less than about 0.1 wt %).

In one embodiment, the composition is free from or substantially free from MCTs comprising fatty acid moieties that are not decanoic acid and octanoic acid. In one embodiment, the composition is free from or substantially free from MCTs comprising fatty acid moieties that are not decanoic acid. However, there may be traces of such MCTs in the composition (e.g., less than about 5 wt %, less than about 4 wt %, less than about 3 wt %, less than about 2 wt %, less than about 1 wt %, less than about 0.5 wt % or less than about 0.1 wt %).

The composition may also comprise long chain triglycerides (LCTs). Preferably the LCTs are present in the composition in amount of less than about 5 wt %, less than about 4 wt %, less than about 3 wt %, less than about 2 wt %, less than about 1 wt %, less than about 0.5 wt % or less than about 0.1 wt %). In one embodiment, no LCTs are present in the composition.

In one embodiment, the composition is substantially free of any other saturated fatty acid. However, there may be traces of other saturated fatty acids in the composition (e.g., less than about 5 wt %, less than about 4 wt %, less than about 3 wt %, less than about 2 wt %, less than about 1 wt %, less than about 0.5 wt % or less than about 0.1 wt %).

In one embodiment, the composition is substantially free of mono- or poly-unsaturated fatty acids. However, there may be traces of mono- or poly-unsaturated fatty acids in the composition (e.g., less than about 5 wt %, less than about 4 wt %, less than about 3 wt %, less than about 2 wt %, less than about 1 wt %, less than about 0.5 wt % or less than about 0.1 wt %).

In one embodiment, the composition comprises pure or substantially pure decanoic acid.

The composition may comprise lipids in an amount of about 60 g to about 80 g, about 63 g to about 75 g, or about 65 g to about 72 g per 100 g dry mass. In one embodiment, the composition comprises lipids in an amount of about 30 g to about 40 g, about 31 g to about 38 g, about 32 g to about 36 g, or about 33 g to about 35 g per 100 mL. In one embodiment the composition comprises lipids in an amount of about 34 g per 100 mL. The composition may comprise MCT in an amount of about 30 g to about 40 g, about 31 g to about 38 g, about 32 g to about 36 g, or about 33 g to about 34 g per 100 mL. In one embodiment the composition comprises MCT in an amount of about 33.5 g per 100 mL.

Coconut oil or palm oil may be a preferred source for at least 50 wt %, or from about 70 wt % to about 90 wt % of the lipids. The remainder of the lipids can be selected from e.g. medium-chain triglyceride sources such as fractionated coconut oil, macadamia oil, palm oil or palm kernel oil, or long-chain triglyceride sources such as safflower oil, sesame seed oil, soy oil (which may be obtained from soybean), sunflower oil, high oleic sunflower oil, corn oil, canola oil, walnut oil, evening primrose oil, peanut oil, cottonseed oil, rapeseed oil, olive oil, fish oil, palm olein or algal oil, or mixtures thereof, preferably soybean oil (suitably between 2 wt % and 30 wt %), medium-chain triglycerides (suitably between 0 wt % and 14 wt %), marine oils (suitably between 0 wt % and 14 wt %, or between 2 wt % and 12 wt %), and phospholipids, mono- and di-glycerides.

The composition may comprise carbohydrate in an amount of about 0 g to about 9 g, about 3.2 g to about 9 g, about 4 g to about 8.6 g, or about 5 g to about 8.2 g per 100 g dry mass. In one embodiment, the composition comprises carbohydrate in an amount of about 0 g to about 1 g per 100 mL, about 0.1 g to about 0.8 g per 100 mL, about 0.2 g to about 0.6 g per 100 mL, or about 0.3 g to about 0.5 g per 100 mL. In one embodiment, the composition comprises carbohydrate in an amount of about 0.42 g per 100 mL.

The composition may comprise protein in an amount of about 5 g to about 20 g, about 13 g to about 20 g, about 13 g to about 18 g, about 13.8 g to about 17 g, or about 14.2 g to about 16.2 g per 100 g dry mass. In one embodiment, the composition comprises protein in an amount of about 0 g to about 1 g per 100 mL, about 0 g to about 0.5 g per 100 mL, about 0 g to about 0.2 g per 100 mL, or about 0 g to about 0.1 g per 100 mL. In one embodiment, the composition comprises protein in an amount of about 0 g per 100 mL. The inclusion of alpha-lactalbumin or ingredients which comprise high amounts of protein, are particularly suitable. The presence of more than 20 wt % alpha-lactalbumin in the protein fraction of the product results in easy compliance with the requirements for leucine, lysine, methionine and cysteine, excellent palatability and digestion properties. In one embodiment, more than about 20 wt % or about 40 to about 80 wt % of the protein fraction is alpha-lactalbumin.

In another embodiment, the composition is free, or substantially free, of carbohydrate and protein, e.g. the composition has less than 2 wt %, 1 wt %, 0.5 wt % or 0.1 wt % carbohydrate and protein by weight. In one embodiment, the weight amounts of lipid to the sum of proteins and carbohydrates in the composition is about 1-5 to 1, or more. For example, the weight amounts of lipid to the sum of proteins and carbohydrates may be about 1:1, about 2:1, about 3:1, about 4:1, about 5:1, about 2.0-5.0 to 1, about 2.4-4.0 to 1, or about 2.6-3.8 to 1, or more.

The composition may further comprise substances such as minerals, vitamins, salts, functional additives including, for example, palatants, colorants, emulsifiers, antimicrobial or other preservatives. In one embodiment, the composition further comprises at least one substance selected from the group consisting of mineral, vitamins, salts, preservatives, and combinations thereof. Minerals that may be useful in such compositions include, for example, calcium, phosphorous, potassium, sodium, iron, chloride, boron, copper, zinc, magnesium, manganese, iodine, selenium, chromium, molybdenum, fluoride and the like. Examples of vitamins that may be useful in compositions described herein include water soluble vitamins (such as 12hiamine (vitamin B1), riboflavin (vitamin B2), niacin (vitamin B3), pantothenic acid (vitamin B5), pyridoxine (vitamin B6), biotin (vitamin B7), myo-inositol (vitamin B8), folic acid (vitamin B9), cobalamin (vitamin B12), and vitamin C) and fat soluble vitamins (such as vitamin A, vitamin D, vitamin E, and vitamin K) including salts, esters or derivatives thereof. Inulin, taurine, carnitine, amino acids, enzymes, coenzymes, and the like may be useful to include in various embodiments. In one embodiment, the composition comprises salt in an amount of about 0.0 to about 0.2 g per 100 mL In one embodiment, the composition comprises salt in an amount of about 0.11 g per 100 mL. Preservatives, stabilisers, dyes and even flavouring agents may be provided in the composition. Examples of preservatives include sodium benzoate, sorbic acid and esters of p hydroxybenzoic acid. Antioxidants and suspending agents may be also used.

The composition may be relatively energy dense. In one embodiment, the composition comprises about 2520 to about 3780, about 2520 to about 3080, or about 2800 to about 3040 kJ per 100 grams dry matter. The composition may comprise about 2500 to about 3100 kJ per 100 grams dry matter, such as about 2505, about 2510, about 2515, about 2520, about 2525, about 2530, about 2535 or about 2540 to about 3100, about 3095, about 3090, about 3085 or about 3080 kJ per 100 g. In one embodiment, the composition comprises about 2984, about 2985, about 2986, about 2987, about 2988, about 2989 or about 2990 KJ per 100 g. In one embodiment, the composition comprises about 2500 to about 3100 kJ per 100 grams dry matter. In one embodiment, the composition comprises about 2540 to about 3080 kJ per 100 grams dry matter. In one embodiment, the composition comprises about 1000 to about 1500, about 1100 to about 1400, or about 1200 to about 1300 kJ per 100 mL. In one embodiment, the composition comprises about 1265 kJ per 100 mL.

The composition may have a ketogenic ratio of from about 0.2 to about 0.3:1. Other embodiments include, but are not limited to, ratios of about 0.5:1, about 1:1 to about 5:1, up to about 4:1, optionally wherein the majority of fat is decanoic acid. In one embodiment, the composition has a ketogenic ratio of about 2:1 to about 4:1. In one embodiment, the composition has a ketogenic ratio of about 2.7:1 to about 3.4:1.

### Exemplary Composition

An exemplary composition is shown in the table below. In one embodiment, the composition is a ready-to-drink oil-in-water emulsion and comprises the following:

| | | per 100 mL | per 120 mL pack |
|---|---|---|---|
| Energy | kJ | 1265 | 1518 |
| Fat | g | 34 | 41 |
| of which saturates | g | 31 | 37 |
| MCT | g | 33.5 | 40 |
| Decanoic acid | g | 24.5 | 29 |
| Carbohydrate | g | 0.42 | 0.50 |
| Protein | g | 0 | 0 |
| Salt | g | 0.11 | 0.13 |

### Peroxisomal Disorders

In one aspect, the present invention provides decanoic acid, or a composition comprising the same, for use in treating and/or preventing a peroxisomal disorder.

In one aspect, the present invention provides a method of treating and/or preventing a peroxisomal disorder, the method comprising administering a therapeutically effective amount of decanoic acid, or a composition comprising the same, to a subject in need thereof.

In one aspect, the present invention provides the use of decanoic acid, or a composition comprising the same, for the manufacture of a medicament for the treatment and/or prevention of a peroxisomal disorder.

As used herein, the term “treating” may refer to preventing, lessening, reducing or improving at least one symptom associated with the condition and/or slowing down, reducing or blocking the progression of the condition in a subject having one or more symptom the condition.

As used herein, the term “preventing” may refer to reducing or preventing development of at least one symptom associated with the condition in a subject not showing any symptoms of the condition.

Peroxisomes are dynamic organelles that play an essential role in a variety of cellular catabolic and anabolic metabolic pathways, including fatty acid alpha- and beta-oxidation, and plasmalogen and bile acid synthesis. Defects in genes encoding peroxisomal proteins can result in a large variety of peroxisomal disorders either affecting specific metabolic pathways, i.e., the single peroxisomal enzyme deficiencies, or causing a generalized defect in function and assembly of peroxisomes, i.e., peroxisome biogenesis disorders (Waterham, H. R., et al., 2016. Biochimica et Biophysica Acta (BBA)-Molecular Cell Research, 1863 (5), pp. 922-933).

The decanoic acid may improve peroxisomal function and/or peroxisomal number. Any suitable assay may be used to determine the effect on peroxisomal function and/or peroxisomal number. For example, peroxisomal function and/or number may be determined by gene expression analysis of genes encoding peroxisomal proteins or by direct measurements of peroxisomal proteins (see e.g. Huang, T. Y., et al., 2019. Metabolism, 98, pp. 53-61. Peroxisomal function may also be determined by measuring the levels of substrates normally handled by peroxisomes, such as VLCFAs, pristanic acid, phytanic acid, DHCA, THCA, and pipecolic acid and/or the levels of end products of peroxisomal metabolism, such as plasmalogens, cholic and chenodeoxycholic acid, and docosahexaenoic acid (see e.g. Ferdinandusse, S., et al., 2016. Journal of the Society for the Study of Inborn Errors of Metabolism, 39 (4), pp. 531-543). Peroxisomal number may also be determined by immunofluorescence and peroxisome counting as described by e.g. Colton, H. M., et al., 2004. Toxicological Sciences, 80 (1), pp. 183-192. Peroxisomes may be stained e.g. with a polyclonal antibody against a peroxisomal membrane protein and peroxisome number per cell determined manually or using automated software.

The decanoic acid may improve peroxisomal function. In some embodiments, the decanoic acid increases acyl CoA oxidase activity, increases Pex11a expression, and/or increases peroxisomal beta oxidation capacity. In some embodiments, the decanoic acid increases acyl CoA oxidase activity by at least 50% or at least 100%. In some embodiments, the decanoic acid increases PEX11a expression by at least 10%, at least 20%, or at least 30%. In some embodiments, the decanoic acid increases peroxisomal beta oxidation capacity by at least 10%, at least 20%, or at least 30%.

The peroxisomal disorder treated or prevented in the present invention may be a peroxisome biogenesis disorder (PBD) or a peroxisomal enzyme deficiency (PED).

### Peroxisome Biogenesis Disorders (PBDs)

In some embodiments, the peroxisomal disorder is a peroxisome biogenesis disorder (PBD).

Peroxisome biogenesis disorders (PBDs) are a group of autosomal recessive disorders comprised of three distinct subtypes are recognized among the PBDs: the Zellweger spectrum disorders (ZSDs), RCDP type 1 and type 5, and the peroxisomal fission defects. PBDs may be caused by defects in any of at least 14 different PEX genes, which encode proteins involved in peroxisome assembly and proliferation. Genetic testing for all PEX genes is available as diagnostic service (Waterham, H. R. and Ebberink, M. S., 2012. Biochimica et Biophysica Acta (BBA)-Molecular Basis of Disease, 1822 (9), pp. 1430-1441; and Waterham, H. R., et al., 2016. Biochimica et Biophysica Acta (BBA)-Molecular Cell Research, 1863 (5), pp. 922-933).

In some embodiments, the peroxisomal disorder is a Zellweger spectrum disorder (ZSD), RCDP type 1, RCDP type 5 or a peroxisomal fission defect.

### Zellweger Spectrum Disorders (ZSDs)

In some embodiments, the peroxisomal disorder is a Zellweger spectrum disorder (ZSD).

The ZSDs include the three historically defined clinical entities cerebro-hepato-renal syndrome or Zellweger syndrome (ZS), neonatal adrenoleukodystrophy (NALD), and infantile Refsum disease (IRD), which presently are considered different presentations within the same clinical and biochemical spectrum, with ZS being the most and IRD a less severe presentation (Waterham, H. R., et al., 2016. Biochimica et Biophysica Acta (BBA)-Molecular Cell Research, 1863 (5), pp. 922-933). The ZSDs may also include Heimler syndrome (HS) which is caused by hypomorphic mutations in PEX1 and PEX6 (Ratbi, I., et al., 2015. The American Journal of Human Genetics, 97 (4), pp. 535-545).

In some embodiments, the peroxisomal disorder is Heimler syndrome (HS), infantile Refsum disease (IRD), neonatal adrenoleukodystrophy (NALD), or Zellweger syndrome (ZS). In some embodiments, the peroxisomal disorder is HS, IRD or NALD.

### RCDP Type 1 or Type 5

In some embodiments, the peroxisomal disorder is RCDP type 1 or RCDP type 5.

RCDP type 1 is caused by mutations in the PEX7 gene, which codes for the PTS2-protein receptor. RCDP type 5 is caused by a frame shift mutation located in the PEX5L-specific exon 9, which results in loss of only the PEX5L isoform, while PEX5S is still expressed (Waterham, H. R., et al., 2016. Biochimica et Biophysica Acta (BBA)-Molecular Cell Research, 1863 (5), pp. 922-933).

### Peroxisomal Fission Defects

In some embodiments, the peroxisomal disorder is a peroxisomal fission defect.

Patients have been reported with mutations in DLP1, Mff, GDAP1, and Pex11ß (Waterham, H. R., et al., 2007. New England Journal of Medicine, 356 (17), pp. 1736-1741; Nasca, A., et al., 2018. Frontiers in genetics, 9, p. 625; Huber, N., et al., 2013. EMBO reports, 14 (6), pp. 545-552; and Ebberink, M. S., et al. 2012. Journal of medical genetics, 49 (5), pp. 307-313).

### Peroxisomal Enzyme Deficiencies (PEDs)

In some embodiments, the peroxisomal disorder is a peroxisomal enzyme deficiency (PED).

Peroxisomal enzyme deficiencies (PEDs) are a group of disorder caused by a defect of a single peroxisomal protein involved in peroxisomal function such as (1) ether phospholipid (plasmalogen) biosynthesis; (2) fatty acid beta-oxidation; (3) peroxisomal alpha-oxidation; (4) glyoxylate detoxification, and (5) H2O2 metabolism (Wanders, R. J. and Waterham, H. R., 2006. Biochimica et Biophysica Acta (BBA)-Molecular Cell Research, 1763 (12), pp. 1707-1720; and Waterham, H. R., et al., 2016. Biochimica et Biophysica Acta (BBA)-Molecular Cell Research, 1863 (5), pp. 922-933)

In some embodiments, the peroxisomal disorder is a disorder of peroxisomal fatty acid beta-oxidation, a disorder of peroxisomal fatty acid alpha-oxidation, a disorder of peroxisomal glyoxylate metabolism, a disorder of peroxisomal ether phospholipid biosynthesis, a disorder of peroxisomal bile acid synthesis, or a disorder of peroxisomal H₂O₂ metabolism.

In some embodiments, the peroxisomal disorder is x-linked adrenoleukodystrophy (X-ALD); acyl-CoA oxidase deficiency; D-bifunctional protein deficiency; SCPx deficiency; 2-methylacyl-CoA racemace (AMACR) deficiency; Refsum disease; primary hyperoxaluria type 1; RCDP type 2; RCDP type 3; RCDP type 4; ABCD3 deficiency; BAAT deficiency; or Acatalasemia.

### Disorders of Peroxisomal Fatty Acid Beta-Oxidation

In some embodiments, the peroxisomal disorder is a disorder of peroxisomal fatty acid beta-oxidation, such as x-linked adrenoleukodystrophy (X-ALD); acyl-CoA oxidase deficiency; D-bifunctional protein deficiency; SCPx deficiency; or 2-methylacyl-CoA racemace (AMACR) deficiency.

In some embodiments, the peroxisomal disorder is x-linked adrenoleukodystrophy (X-ALD). X-ALD is the most frequently occurring peroxisomal disorder and is caused by mutations in the ABCD1 gene, which encodes one of the three known peroxisomal ABC half transporters located in the peroxisomal membrane, which are involved in the ATP-driven transport of fatty acids across the peroxisomal membrane.

In some embodiments, the peroxisomal disorder is acyl-CoA oxidase deficiency. Acyl-CoA oxidase deficiency is caused by mutations in the ACOX1 gene encoding one of three different peroxisomal acyl-CoA oxidases.

In some embodiments, the peroxisomal disorder is D-bifunctional protein deficiency. D-bifunctional protein deficiency is caused by mutations in the HSD17B4 gene encoding 17-beta-estradiol dehydrogenase, an enzyme catalyzing the second and third step of peroxisomal beta-oxidation.

In some embodiments, the peroxisomal disorder is SCPx deficiency. SCPx deficiency is caused by mutations in the SCP2 gene, which encodes the 58-kDa SCPx protein composed of a 46-kDa thiolase domain and a 13-kDa SCP2 domain.

In some embodiments, the peroxisomal disorder is 2-methylacyl-CoA racemace (AMACR) deficiency. AMACR deficiency is caused by mutations in the AMACR gene encoding the enzyme 2-methylacyl-CoA racemace (AMACR).

### Disorders of Peroxisomal Fatty Acid Alpha-Oxidation

In some embodiments, the peroxisomal disorder is a disorder of peroxisomal fatty acid alpha-oxidation, such as Refsum disease.

In some embodiments, the peroxisomal disorder is Refsum disease. Refsum disease is caused by mutations in the PHYH gene encoding the peroxisomal enzyme phytanoyl-CoA 2-hydroxylase, which catalyzes the first step in the peroxisomal alpha-oxidation pathway.

### Disorders of Peroxisomal Glyoxylate Metabolism

In some embodiments, the peroxisomal disorder is a disorder of peroxisomal glyoxylate metabolism, such as primary hyperoxaluria type 1.

In some embodiments, the peroxisomal disorder is primary hyperoxaluria type 1. Primary hyperoxaluria type 1 is caused by mutations in the AGXT gene encoding the liver-specific peroxisomal enzyme alanine-glyoxylate aminotransferase (AGT), a pyridoxal 5′-phosphate-dependent enzyme that catalyzes the transamination of l-alanine and glyoxylate to pyruvate and glycine.

### Disorders of Peroxisomal Ether Phospholipid Biosynthesis

In some embodiments, the peroxisomal disorder is a disorder of peroxisomal ether phospholipid biosynthesis, such as RCDP type 2, RCDP type 3, or RCDP type 4.

In some embodiments, the peroxisomal disorder is RCDP type 2. RCDP type 2 is also known as GNPAT deficiency and is caused by mutations in the GNPAT gene encoding GNPAT (DHAPAT), the enzyme which catalyzes the first of the two subsequent peroxisomal conversions in the synthesis of ether phospholipids.

In some embodiments, the peroxisomal disorder is RCDP type 3. RCDP type 3 is also known as AGPS deficiency and is caused by mutations in the AGPS gene encoding AGPS, the enzyme which catalyzes the second peroxisomal conversion in the synthesis of ether phospholipids.

In some embodiments, the peroxisomal disorder is RCDP type 4. RCDP type 4 is also known as FAR1 deficiency and may be caused by mutations in the FAR1 gene encoding fatty acyl-CoA reductase 1, which converts fatty acyl-CoAs into their respective fatty alcohol.

### Disorders of Peroxisomal Bile Acid Synthesis

In some embodiments, the peroxisomal disorder is a disorder of peroxisomal bile acid synthesis, such as ABCD3 deficiency or BAAT deficiency.

In some embodiments, the peroxisomal disorder is ABCD3 deficiency. ABCD3 deficiency may be caused by a truncating mutation in the ABCD3 gene. The peroxisomal half ABC transporter ABCD3 is involved in transport of branched-chain fatty acids and C27 bile acids into the peroxisome.

In some embodiments, the peroxisomal disorder is BAAT deficiency. BAAT deficiency is caused by mutations in the BAAT gene encoding the liver-specific peroxisomal enzyme Bile acid-CoA: amino acid N-acyltransferase, which converts the CoA esters of bile acids into taurine or glycine conjugates.

Disorders of Peroxisomal H₂O₂ Metabolism

In some embodiments, the peroxisomal disorder is a disorder of peroxisomal H₂O₂ metabolism, such as Acatalasemia.

In some embodiments, the peroxisomal disorder is Acatalasemia. Acatalasemia is caused by mutations in the CAT gene that code for the prototypical peroxisomal enzyme catalase, which breaks down H₂O₂ into H₂O and O₂.

### Subject

The agents and methods may be used to treat or prevent a peroxisomal disorder in any subject in need thereof.

The subject may be a human or an animal. The subject may be a mammal such as a human, canine, feline, equine, caprine, bovine, ovine, porcine, cervine and primates. In preferred embodiments, the subject is a human. In other embodiments, the subject is an animal, preferably wherein the animal is a pet. A pet may be an animal selected from dogs, cats, birds, fish, rodents such as mice, rats, and guinea pigs, rabbits, etc.

The subject may be any age. Suitably, the subject may be a child or an adult. In some embodiments, the subject is a juvenile, an adolescent, a child, or an infant. The term “juvenile” may refer to an individual that has not yet reached adulthood. The term “adolescent” may refer to an individual during the period from the onset of puberty to adulthood. The term “child” may refer an individual between the stages of birth and puberty. The term “infant” may refer to a human subject aged from about 0 years to about 1 year. In other embodiments, the subject is an adolescent or an adult. In one embodiment, the subject is a human aged about 3 years or older.

The subject may have any peroxisomal disorder described herein. Any suitable diagnostic method may be used to determine if the subject has a peroxisomal disorder (see e.g. Ferdinandusse, S., et al., 2016. Journal of the Society for the Study of Inborn Errors of Metabolism, 39 (4), pp. 531-543; and Klouwer, F. C., et al., 2016. Neuropediatrics, 47 (04), pp. 205-220). For example, a peroxisomal disorder can be confirmed by biochemical testing and/or genetic testing.

The subject may have increased plasma levels of substrates normally handled by peroxisomes, such as VLCFAs, pristanic acid, phytanic acid, DHCA, THCA, and pipecolic acid. The subject may have decreased levels of end products of peroxisomal metabolism, such as plasmalogens, cholic and chenodeoxycholic acid, and docosahexaenoic acid.

The subject may have a defect in a PEX gene, a gene associated with a peroxisomal fission defect and/or a gene encoding a peroxisomal protein. As described above, PBDs may be caused by defects in any of at least 14 different PEX genes, which encode proteins involved in peroxisome assembly and proliferation and PEDs a caused by defects in any peroxisomal protein involved in peroxisomal function. The development of next generation sequencing technology enables parallel sequencing of multiple genes involved in peroxisomal disorders. PBDs may be diagnosed by PEX cDNA transfection complementation assays followed by sequencing of the implicated PEX gene (see e.g. Krause, C., et al., 2009. European journal of human genetics, 17 (6), pp. 741-748).

In some embodiments, the subject has a defect in one or more gene selected from: PEX1, PEX2, PEX3, PEX5, PEX6, PEX7, PEX10, PEX11a, PEX11B, PEX11y, PEX12, PEX13, PEX14, PEX16, PEX19, PEX26, DLP1, Mff, GDAP1, Fis1, ABCD1, ACOX1, HSD17B4, SCP2, AMACR, PHYH, AGXT, GNPAT, AGPS, FAR1, ABCD3, BAAT, and CAT.

In some embodiments, the subject has a defect in one or more PEX gene. In some embodiments, the subject has a defect in one or more gene selected from: PEX1, PEX2, PEX3, PEX5, PEX6, PEX7, PEX10, PEX11a, PEX11B, PEX11y, PEX12, PEX13, PEX14, PEX16, PEX19, and PEX26.

In some embodiments, the subject has a defect in one or more gene associated with a PBD.

In some embodiments, the subject has a defect in one or more gene associated with a peroxisomal fission defect. In some embodiments, the subject has a defect in one or more gene selected from: DLP1, Mff, GDAP1, Fis1, PEX11a, PEX11B, and PEX11y.

In some embodiments, the subject has a defect in one or more gene encoding a peroxisomal protein. In some embodiments, the subject has a defect in one or more gene selected from: ABCD1, ACOX1, HSD17B4, SCP2, AMACR, PHYH, AGXT, GNPAT, AGPS, FAR1, ABCD3, BAAT, and CAT.

### Administration

The decanoic acid may be administered to the subject by any suitable route. The decanoic acid or composition comprising the same may administered enterally or parenterally.

In some embodiments, the decanoic acid or composition comprising the same is administered enterally. Enteral administration may be oral, gastric, and/or rectal. In general terms, administration of the combination or composition described herein may, for example, be by an oral route or another route into the gastro-intestinal tract, for example the administration may be by tube feeding. In some embodiments, the decanoic acid or composition comprising the same is administered orally or via feeding tube.

The decanoic acid or composition comprising the same may be administered to the subject via any suitable administration regime. Suitably, the decanoic acid or composition comprising the same is administered once a day, twice a day, three times a day, or four times a day. Suitably, the decanoic acid or composition comprising the same is administered as part of a meal, snack or enteral feed. Suitably, the decanoic acid or composition comprising the same is administered daily for at least six days.

The decanoic acid or composition comprising the same may be administered to the subject in any suitable dosage. Suitably, the decanoic acid is administered in an amount of at least about 2 g, at least about 5 g per day, or at least about 10 g per day. Suitably, the decanoic acid is administered in an amount of about 2 g to about 200 g per day, about 5 g to about 150 g per day, or about 10 g to about 100 g per day. In one embodiment, the decanoic acid is administered in an amount of about 14.5 to about 58 g per day. In one embodiment, the decanoic acid is administered in an amount of about 14.5 g per day, about 29 g per day, or about 58 g per day.

In one embodiment, the decanoic acid or composition comprising the same is administered as part of a ketogenic diet. If the decanoic acid or composition comprising the same is administered as part of a ketogenic diet, the ratio of total fat content:protein/carbohydrate content can be altered during therapy to achieve nutritional goals and to optimise clinical benefit.

The classical version of the ketogenic diet uses ratios to determine and describe fat content. The ketogenic ratio represents the relationship between the grams of fat and the combined grams of protein and carbohydrate. In a 4:1 ratio there are four times as many grams of fat for every 1 g of protein and carbohydrate combined. The ratio is traditionally intended to regulate the degree of ketosis, with higher ratios theoretically stimulating greater ketosis. The ketogenic ratio can be in the range of, for example about 1:1 to about 7:1, about 1:1 to about 5:1, for example, about 1:1, about 1.5:1, about 2:1, about 2.5:1, about 3:1, about 3.5:1, about 4:1, about 4.5:1 or about 5:1. In one embodiment the ketogenic ratio is about 2.25:1 to about 3.9:1. In another embodiment, the ketogenic ratio is about 2.26 to about 3.8:1 or about 2.7 to about 3.4:1. In further embodiments, the ketogenic ratio is about 3.21:1, about 3.23:1, about 3.24:1, about 3.25:1, about 3.26:1, about 3.27:1, about 3.28:1 or about 3.29:1.

### Peroxisome Proliferators

In one aspect, the present invention provides use of decanoic acid as a peroxisome proliferator. The use may be in vitro. As used herein, a “peroxisome proliferator” may refer to an agent which induces peroxisome proliferation

### EXAMPLES

The invention will now be further described by way of Examples, which are meant to serve to assist one of ordinary skill in the art in carrying out the invention and are not intended in any way to limit the scope of the invention.

### Example 1—Decanoic Acid and Peroxisomal Function

SH-SY5Y cells were stored frozen and cells with passage numbers between 20 and 24 were utilized. When required, SH-SY5Y cells were thawed at 20-25° C. and quickly seeded at a density of 13.3×10³ cell/cm² in 75 cm² flasks. SH-SY5Y cells were then grown in media containing 1:1 DMEM/F-12+100 mL/L FBS+10 mL/L I-glutamine at +37° C. and 5% CO₂. SH-SY5Y cells were exposed to 250 μM decanoic acid (C10) or octanoic acid (C8) for 6 days.

To assess effects upon peroxisomal content and function, the following parameters were studied; (a) activity of the peroxisomal marker enzyme, acyl CoA oxidase, (b) expression of Pex11a, a gene associated with peroxisomal biogenesis and (c) evaluation of peroxisomal fatty acid beta oxidation by monitoring the chain shortening of a very long chain fatty acid, C22, to C18, C16 and C14.

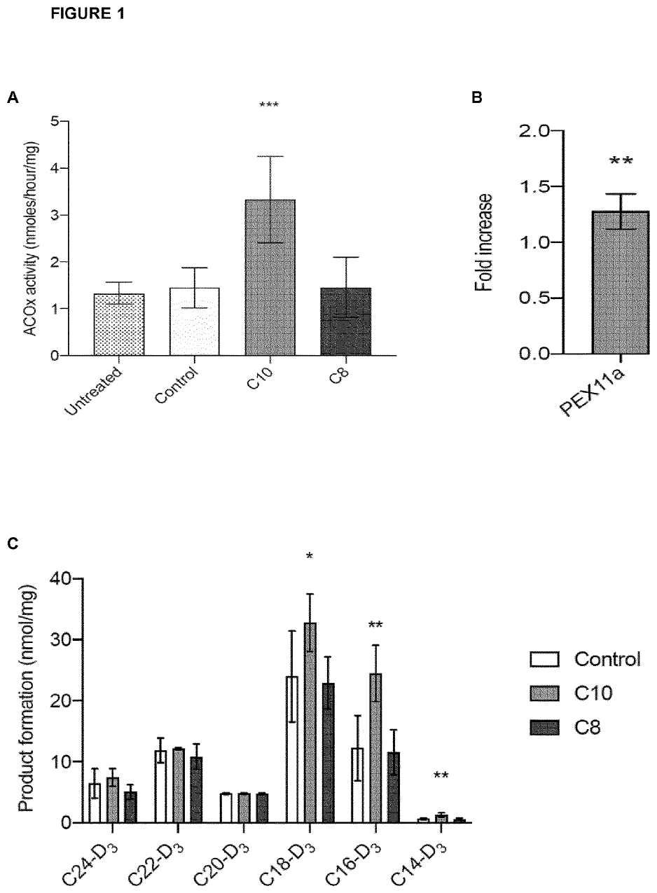

C10 exposure was associated with an increase in acyl CoA oxidase activity (FIG. 1A), increased Pex11a (FIG. 1B) and increased capacity to perform peroxisomal beta oxidation (FIG. 1C). These results show that C10 may exert additional positive effects upon peroxisomal function.

### EMBODIMENTS

Various preferred features and embodiments of the present invention will now be described with reference to the following numbered paragraphs (paras).
1. Decanoic acid for use in treating and/or preventing a peroxisomal disorder in a subject.
2. Decanoic acid for use according to para 1, wherein the peroxisomal disorder is a peroxisome biogenesis disorder (PBD) or a peroxisomal enzyme deficiency (PED).
3. Decanoic acid for use according to para 1 or 2, wherein the peroxisomal disorder is a Zellweger spectrum disorder (ZSD); rhizomelic chondriodysplasia punctata (RCDP) type 1; RCDP type 5; x-linked adrenoleukodystrophy (X-ALD); acyl-CoA oxidase deficiency; D-bifunctional protein deficiency; SCPx deficiency; 2-methylacyl-CoA racemace (AMACR) deficiency; Refsum disease; primary hyperoxaluria type 1; RCDP type 2; RCDP type 3; RCDP type 4; ABCD3 deficiency; BAAT deficiency; or Acatalasemia.
4. Decanoic acid for use according to any preceding para, wherein the peroxisomal disorder is a peroxisome biogenesis disorder (PBD).
5. Decanoic acid for use according to any preceding para, wherein the peroxisomal disorder is a Zellweger spectrum disorder (ZSD), rhizomelic chondriodysplasia punctata (RCDP) type 1, or RCDP type 5, optionally wherein the peroxisomal disorder is a ZSD.
6. Decanoic acid for use according to any preceding para, wherein the peroxisomal disorder is Heimler syndrome (HS), infantile Refsum disease (IRD), neonatal adrenoleukodystrophy (NALD), or Zellweger syndrome (ZS), optionally wherein the peroxisomal disorder is HS, IRD or NALD.
7. Decanoic acid for use according to any preceding para, wherein the subject has a defect in a PEX gene or a gene encoding a peroxisomal protein.
8. Decanoic acid for use according to any preceding para, wherein the subject has a defect in one or more gene selected from: PEX1, PEX2, PEX3, PEX5, PEX6, PEX7, PEX10, PEX11B, PEX12, PEX13, PEX14, PEX16, PEX19, PEX26, ABCD1, ACOX1, HSD17B4, SCP2, AMACR, PHYH, AGXT, GNPAT, AGPS, FAR1, ABCD3, BAAT, and CAT.
9. Decanoic acid for use according to any preceding para, wherein the subject has a defect in a PEX gene.
10. Decanoic acid for use according to any preceding para, wherein the subject has a defect in one or more gene selected from: PEX1, PEX2, PEX3, PEX5, PEX6, PEX7, PEX10, PEX11B, PEX12, PEX13, PEX14, PEX16, PEX19, PEX26.
11. Decanoic acid for use according to any preceding para, wherein the subject is a mammal, preferably wherein the subject is a human.
12. Decanoic acid for use according to any preceding para, wherein the subject is a child or an adult.
13. Decanoic acid for use according to any preceding para, wherein the decanoic acid is in the form of triglycerides, diacyl-glycerides, monoacyl-glycerides, a pharmaceutically acceptable salt, and/or an ester.
14. Decanoic acid for use according to any preceding para, wherein the decanoic acid is in the form of triglycerides, diacyl-glycerides, and/or monoacyl-glycerides.
16. Decanoic acid for use according to any preceding para, wherein the decanoic acid is in the form of triglycerides, preferably wherein the decanoic acid is in the form of medium-chain triglycerides.
17. Decanoic acid for use according to any preceding para, wherein the decanoic acid is in the form of a C10 homotriglyceride.
18. Decanoic acid for use according to any preceding para, wherein the decanoic acid is comprised in a composition.
19. Decanoic acid for use according to para 18, wherein the composition is a pharmaceutical composition, a nutritional composition, or a nutritional supplement.
20. Decanoic acid for use according to para 18 or 19, wherein the composition is in the form of an oil-in-water emulsion, a powder, or a foodstuff, preferably wherein the composition is in the form of an oil-in-water emulsion.
21. Decanoic acid for use according to any of paras 18 to 20, wherein the composition is in a ready-to-use form.
22. Decanoic acid for use according to any of paras 18 to 21, wherein the composition is free from or substantially free from fatty acid moieties that are not decanoic acid or octanoic acid, optionally wherein the composition is free from or substantially free from fatty acid moieties that are not decanoic acid.
23. Decanoic acid for use according to any of paras 18 to 22, wherein the composition is free from MCTs comprising fatty acid moieties that are not decanoic acid or octanoic acid, optionally wherein the composition is free from or substantially free from MCTs comprising fatty acid moieties that are not decanoic acid.
24. Decanoic acid for use according to any of paras 18 to 23, wherein the composition comprises proteins, fat and carbohydrates, and wherein at least about 50 wt % of the fat is decanoic acid, optionally wherein at least about 55 wt %, at least about 60 wt %, at least about 65 wt %, at least about 70 wt % of the fat is decanoic acid.
25. Decanoic acid for use according to any of paras 18 to 24, wherein the decanoic acid is present in the composition in an amount of at least about 100 g/L, optionally wherein the decanoic acid is present in the composition in an amount of at least about 110 g/L, at least about 120 g/L, at least about 130 g/L, at least about 140 g/L, at least about 150 g/L, at least about 160 g/L, at least about 180 g/L, at least about 200 g/L, at least about 220 g/L, or at least about 240 g/L.
26. Decanoic acid for use according to any preceding para, wherein the decanoic acid is administered enterally or parenterally, preferably wherein the decanoic acid is administered enterally.
27. Decanoic acid for use according to any preceding para, wherein the decanoic acid is administered orally or via feeding tube.
28. Decanoic acid for use according to any preceding para, wherein the decanoic acid is administered once a day, twice a day, three times a day, or four times a day.
29. Decanoic acid for use according to any preceding para, wherein the decanoic acid is administered as part of a meal, snack or enteral feed.
30. Decanoic acid for use according to any preceding para, wherein the decanoic acid is administered in an amount of about 2 g to about 200 g per day, about 5 g to about 150 g per day, or about 10 g to about 100 g per day.
31. Decanoic acid for use according to any preceding para, wherein the decanoic acid improves peroxisomal function and/or peroxisomal number.
32. Decanoic acid for use according to any preceding para, wherein the decanoic acid improves peroxisomal function.
33. Decanoic acid for use according to any preceding para, wherein the decanoic acid increases acyl CoA oxidase activity, increases Pex11a expression, and/or increases peroxisomal beta oxidation capacity.
34. A method of treating and/or preventing a peroxisomal disorder, the method comprising administering decanoic acid to a subject in need thereof.
35. Use of decanoic acid for the manufacture of a medicament for the treatment and/or prevention of a peroxisomal disorder.
36. Use of decanoic acid as a peroxisome proliferator in vitro.

1. A method of treating and/or preventing a peroxisomal disorder in a subject, the method comprising administering decanoic acid to the subject.

2. The method according to claim 1, wherein the peroxisomal disorder is a peroxisome biogenesis disorder (PBD) or a peroxisomal enzyme deficiency (PED).

3. The method according to claim 1, wherein the subject has a defect in a PEX gene or a gene encoding a peroxisomal protein.

4. The method according to claim 1, wherein the decanoic acid is in the form of triglycerides, diacyl-glycerides, monoacyl-glycerides, a pharmaceutically acceptable salt, and/or an ester.

5. The method according to claim 1, wherein the decanoic acid is in the form of a C10 homotriglyceride.

6. The method according to claim 1, wherein the decanoic acid is administered comprised in a composition.

7. The method according to claim 6, wherein the composition is a pharmaceutical composition, a nutritional composition, or a nutritional supplement.

8. The method according to claim 6, wherein the composition is an oil-in-water emulsion, a powder, or a foodstuff.

9. The method according to claim 6, wherein the composition is in a ready-to-use form.

10. The method according to claim 6, wherein the composition comprises proteins, fat and carbohydrates, and wherein at least about 50 wt % of the fat is the decanoic acid.

11. The method according to claim 6, wherein the decanoic acid is present in the composition in an amount of at least about 100 g/L.

12. The method according to claim 1, wherein the decanoic acid is administered enterally or parenterally.

13. The method according to claim 1, wherein the decanoic acid is administered once a day, twice a day, three times a day, or four times a day.

14. The method according to claim 1, wherein the decanoic acid is administered as part of a meal, snack or enteral feed.

15. The method according to claim 1, wherein the decanoic acid improves peroxisomal function and/or peroxisomal number.

16. The method according to claim 1, wherein the peroxisomal disorder is a Zellweger spectrum disorder (ZSD); rhizomelic chondriodysplasia punctata (RCDP) type 1; RCDP type 5; x-linked adrenoleukodystrophy (X-ALD); acyl-CoA oxidase deficiency; D-bifunctional protein deficiency; SCPx deficiency; 2-methylacyl-CoA racemace (AMACR) deficiency; Refsum disease; primary hyperoxaluria type 1; RCDP type 2; RCDP type 3; RCDP type 4; ABCD3 deficiency; BAAT deficiency; or Acatalasemia.

17. The method according to claim 1, wherein the subject has a defect in one or more genes selected from the group consisting of PEX1, PEX2, PEX3, PEX5, PEX6, PEX7, PEX10, PEX11B, PEX12, PEX13, PEX14, PEX16, PEX19, PEX26, ABCD1, ACOX1, HSD17B4, SCP2, AMACR, PHYH, AGXT, GNPAT, AGPS, FAR1, ABCD3, BAAT, and CAT.

18. The method according to claim 6, wherein the composition is an oil-in-water emulsion.

19. The method according to claim 6, wherein the composition comprises proteins, fat and carbohydrates, and wherein the decanoic acid is at least about 70 wt % of the fat.

20. The method according to claim 6, wherein the decanoic acid is present in the composition in an amount of at least about 240 g/L.
